(19) United States
(12) Patent Application Publication
(10) Pub. No.: US 2026/0315134 A1
(43) Pub. Date: Oct. 15, 2026

(54) METHOD FOR ABLATING TISSUE USING A MULTI-MODE ELECTROSURGICAL DEVICE

(71) Applicant: Baylis Medical Technologies Inc., Mississauga (CA)

(72) Inventors: Neil Godara, Milton (CA); Abelardo Escoto, Toronto (CA); Christopher James Breadner, Ajax (CA); Emily Linqing Ma, Windsor (CA)

(21) Appl. No.: 19/575,275

(22) Filed: Mar. 23, 2026

Related U.S. Application Data
(60) Provisional application No. 63/775,406, filed on Mar. 21, 2025.

(51) Int. Cl. A61B 18/12; A61B 18/00; A61B 18/14; A61B 18/16

(52) U.S. Cl. CPC A61B 18/1206; A61B 18/148; A61B 18/16; A61B 2018/00023; A61B 2018/00327; A61B 2018/00577; A61B 2018/00797; A61B 2018/00821; A61B 2018/1253; A61B 2018/126

(57) ABSTRACT

A method and apparatus are disclosed for a bimodal, electrosurgical probe for ablating soft tissue. The electrosurgical probe includes at least first and second electrodes and is operable to switch between a monopolar radiofrequency energy delivery mode and a bipolar radiofrequency energy delivery mode. An exemplary disclosed method of using the probe includes the steps of: advancing the probe into tissue; delivering energy in a first mode; and delivering energy in a second mode, wherein the ablation zone volume varies depending on the mode selected. Some versions of the probe include internal cooling. Some versions of the method further include the steps of monitoring the temperature of tissue to which the energy is being delivered; and controlling the delivery of energy in response to the temperature of the tissue.

### TECHNICAL FIELD

The disclosure relates to medical devices, and more particularly to devices, systems, and methods for ablating tissue using radiofrequency (RF) energy.

### BACKGROUND OF THE ART

The ablation of benign thyroid nodules has been touted as a possible replacement for surgical resection of the thyroid gland, for symptomatic benign thyroid nodules. While there are several benefits to percutaneous ablation when compared to surgical resection, such as better preservation of thyroid function and improved aesthetic outcomes, there are inherent limitations and risks when using radiofrequency ablation. Thyroid nodules are often located close to sensitive neural structures, such as the recurrent laryngeal nerve. Other structures of interest also surround thyroid nodules (e.g. esophagus, carotid artery, vocal cords) and must also be avoided. When ablating tissue, it may not be possible to predict the extent of the ablation volume. As such, users will often leave a safety margin between where they place the probe tip and the neural structure to be ablated. The size of the safety margin is directly related to the expected ablation volume-the larger the expected ablation volume, the greater the safety margin needs to be.

Because of these requirements, a user must decide between a smaller ablation zone (typically via a small active tip on the RF electrode) which allows a smaller safety margin and therefore better coverage of the overall nodule, but at the cost of reduced procedure efficiency (as more ablations are required to ablate the entire volume of the nodule); or, a larger ablation zone (typically with a larger active tip) which allows for a more efficient procedure but requires a greater safety margin.

These procedures are typically conducted with ultrasound imaging guidance. The ablation is performed from the deeper and medial portions of the nodule towards the more superficial portions of the nodule, using what is called a “moving shot” technique.

An RF ablation device that is configured to produce ablation zones having various sizes could offer advantages.

### BRIEF DESCRIPTION OF THE DRAWINGS

In order that the invention may be readily understood, embodiments of the invention are illustrated by way of examples in the accompanying drawings, in which:

FIG. 1 is an illustration of a system for delivering RF energy to ablate tissue in accordance with an embodiment of the present invention;

FIG. 2 is an illustration of an RF energy delivery device in accordance with an embodiment of the present invention;

FIGS. 3A to 3D are illustrations of the distal region of the device of FIG. 2, and corresponding ablation zones, in accordance with an embodiment of the present invention;

FIGS. 4A to 4C are illustrations of the distal region of an RF device and corresponding ablation zones in accordance with an alternative embodiment of the present invention;

FIG. 5A is a cross-section illustration of the distal region of the RF device in accordance with an embodiment of the present invention;

FIG. 5B is a cross-section illustration of the distal region of the RF device in accordance with an alternative embodiment of the present invention;

FIG. 6 is an illustration of the distal region of the RF device in accordance with an embodiment of the present invention;

FIG. 7 is an illustration of the RF device in accordance with an embodiment of the present invention;

FIG. 8 is an illustration of thyroid anatomy showing the position of the RF device when ablating a thyroid nodule in accordance with an embodiment of the present invention;

FIG. 9 is a flow diagram showing a method of ablating tissue in accordance with an embodiment of the present invention;

FIGS. 10A to 10D illustrate a method of ablating tissue in accordance with an embodiment of the present invention;

FIG. 11 is a chart showing temperature throughout various stages of an ablation in accordance with an embodiment of the present invention; and

FIGS. 12A to 12C are flow charts showing the steps of each stage of an ablation procedure, in accordance with an embodiment of the present invention.

In the drawings, like reference characters generally refer to the same parts throughout the different views. Also, the drawings are not necessarily to scale, emphasis instead being placed upon generally illustrating the various concepts discussed herein.

### DETAILED DESCRIPTION

Current RF devices are configured to have a preset energy delivery profile, usually either monopolar or bipolar. In most applications, this is preferred as it allows the user to select an ablation zone and electrode type that best suits the need for a given ablation target.

In the case of thyroid nodules, the desired ablation zone can be large, but at the same time, can abut or be close to sensitive structures. In those instances, the user must decide on either a smaller, more controlled ablation zone (typically monopolar with a small active tip), or a larger ablation zone (with a bipolar electrode or large monopolar electrode).

The inventors have developed a bimodal probe that allows for both configurations in a single device. When the electrode is placed closed to a sensitive structure, or at any time the user wants a smaller ablation volume, the user can switch the RF energy output profile to a monopolar setting. In one example, this might be the distal 4 mm of the probe, comprising the energy delivering electrode in the monopolar configuration. Once the user has ablated the areas where a smaller ablation is required and has now moved into the “bulk” of the nodule where larger ablation zones would be preferable, the user can switch the probe to a bipolar mode to gain the benefit of large ablation zones and improved procedural efficiency. The system includes at least one switching mechanism to allow a user to control a probe, control energy delivery, and/or change modes/settings of ablation while a procedure is happening. Switching mechanism is a way to control modes of energy delivery, or mode control. In other words modes can be switched mid-procedure rather than set up at the beginning of the procedure. One or more switching mechanisms may have a variety of forms and/or activated a number of ways, such as voice commands, switches, buttons, dials located on the probe, on the generator, a foot pedal, etc. In some embodiments, computers, software, artificial intelligence may be used to control and switch modes during a procedure.

The ability for the user to select the active electrode in situ allows for real-time assessment and evaluation of the nodule with respect to safety concerns.

In one broad aspect, embodiments of the present invention comprise a method of delivering energy to tissue using a radiofrequency probe, the RF probe operable between a plurality of modes, the method comprising the steps of: delivering radiofrequency energy to create a first ablation zone within a region of tissue while the RF probe is in a first mode of the plurality of modes; delivering radiofrequency energy to create a second ablation zone within the region of tissue while the RF probe is in a second mode of the plurality of modes; a first ablation zone volume differs from a second ablation zone volume.

As a feature of this aspect, the first mode is a monopolar mode, and the second mode is a bipolar mode.

As a feature of this aspect, the method further includes a step of cooling a portion of the elongate member.

As a feature of this aspect the method further comprises the steps of: measuring a threshold temperature value, and automatically cooling the portion of the elongate member when the threshold temperature has reached a pre-determined temperature.

As a feature of this aspect, while in the first mode, the energy has a first energy delivery profile and while in the second mode the energy has a second energy delivery profile.

As a feature of this aspect, the first energy delivery profile creates an ablation zone having a first parameter, and the second energy delivery profile creates an ablation zone having a second parameter, the first parameter different from the second parameter, and the first and second parameters are selected from a group consisting of size, form, and location.

As a feature of this aspect, the first mode is a first monopolar mode, and the second mode is a second monopolar mode.

As a feature of this aspect, the first mode is a first bipolar mode, and the second mode is a second bipolar mode.

As a feature of this aspect, the method further comprises a step of selecting an active electrode and selecting a return electrode.

As a feature of this aspect, delivering radiofrequency energy in either mode is performed until a predetermined target parameter has been reached.

As a feature of this aspect, the target parameter is selected from a group consisting of time, impedance, and an amount of joules delivered.

As a feature of this aspect, the method further comprises a step of toggling between the first and second modes at least twice.

As a feature of this aspect, the region of tissue is a thyroid nodule.

As a feature of this aspect, the ablation zone created in one of the first mode or the second mode is located substantially around a perimeter of the thyroid nodule, and the ablation zone created in the other mode is located substantially at a distance from the perimeter/periphery of the thyroid nodule.

In a further broad aspect, embodiments of the present invention comprise: a bi-modal radiofrequency ablation probe capable of operating in a plurality of energy delivery modes, the bi-modal radiofrequency ablation probe operable to be electrically coupled to an external return electrode, the probe comprising: an elongate member; and a plurality of electrodes associated with the elongate member and operable to deliver energy in a plurality of energy delivery modes; the probe is configured to allow a user to select an active electrode from the plurality of electrodes; and the probe is further configured to allow the user to select a return electrode from the group consisting of the plurality of electrodes and the external return electrode.

In a further broad aspect, embodiments of the present invention comprise a bi-modal radiofrequency ablation probe capable of operating in a plurality of energy delivery modes, the bi-modal radiofrequency ablation probe comprising: an elongate member; at least two electrodes associated with the elongate member; and a switch for directing the flow of radiofrequency energy through the at least two electrodes; where, in use, the probe is operable to be electrically coupled to an external return electrode and the switch is operable to direct radiofrequency energy to flow in one of a plurality of energy deliver modes; and in a first mode of the plurality of energy delivery modes, energy is directed to flow between the at least two electrodes associated with the elongate member, and, in a second mode of the plurality of energy delivery modes, energy is directed to flow between one of the at least two electrodes associated with the elongate member and the external return electrode.

As a feature of this aspect, the elongate member defines a distal end and a proximal end, and a lumen defined substantially therebetween, and in use, the lumen is capable of circulating a cooling fluid through a portion of the lumen.

As a feature of this aspect, the probe further comprises a handle.

As a feature of this aspect, the switch is located on the handle.

As a feature of this aspect, the first electrode has a first surface area, and the second electrode has a second surface area, and the first and second surface areas differ.

As a feature of this aspect, the ablation probe is structured to be in communication with a generator and the ablation probe is operable to switch between modes based on instructions from the generator.

As a feature of this aspect, the probe further comprises at least one temperature sensor.

As a feature of this aspect, the at least one temperature sensor is associated with one of the at least two electrodes.

As a feature of this aspect, the ablation probe further comprises at least two temperature sensors.

As a feature of this aspect, one each of the at least two temperature sensors is associated with one electrode of the at least two electrodes.

In a further broad aspect, embodiments of the present invention comprise a system for ablating tissue, the system comprising: a radiofrequency probe operable in a plurality of energy delivery modes and configured to deliver energy to tissue within a patient's body, the radiofrequency probe comprising at least two electrodes; an external return electrode; a user selection switch for allowing a user to select an energy delivery mode from the plurality of energy delivery modes; and a generator electrically coupled to the probe and to the external return electrode, the generator configured to deliver energy to the radiofrequency probe, the generator further comprising a switching mechanism for diverting energy flow between at least two electrodes selected from the group consisting of the at least two electrodes located on the radiofrequency probe and the external return electrode; the radiofrequency energy has a first energy delivery profile while in the first energy delivery mode and a second energy delivery profile in the second energy delivery mode.

As a feature of this aspect, the user selection switch is located on the radiofrequency probe.

As a feature of this aspect, the system further comprises a pump for circulating a cooling fluid within a portion of the radiofrequency probe.

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The inventors have developed a bimodal or multi-mode probe that facilitates both small and large volume ablations in a single device. Specifically, a coaxial bipolar probe is configured with a user-selectable switch that allows the user to select either monopolar or bipolar radiofrequency energy delivery. When the user selects monopolar energy delivery, RF will be delivered between one of the distal or proximal electrodes and a return electrode pad connected to the patient. When the user selects bipolar energy delivery, RF will be delivered between the two electrodes on the shaft of the probe. In some embodiments, a user may select between three modes, including: a first monopolar mode that uses the distal electrode, a second monopolar mode that uses the proximal electrode, and a bipolar mode that uses the distal and proximal electrodes.

The user can switch energy delivery modes in a variety of ways, depending on the complexity of the RF generator. For example, in some configurations, the switch from bipolar to monopolar is done mechanically through a switch that connects either the grounding pad or proximal electrode to the RF return wire that is sent to the generator. In this way, the generator would have no knowledge of the RF return source. In other configurations, the energy delivery output modality is controlled by the RF generator, and the switch is intended only to inform the RF generator of the user's intentions, to which the RF generator modulates the RF return to be either from the cable leading to the probe (bipolar) or the return electrode pad (monopolar).

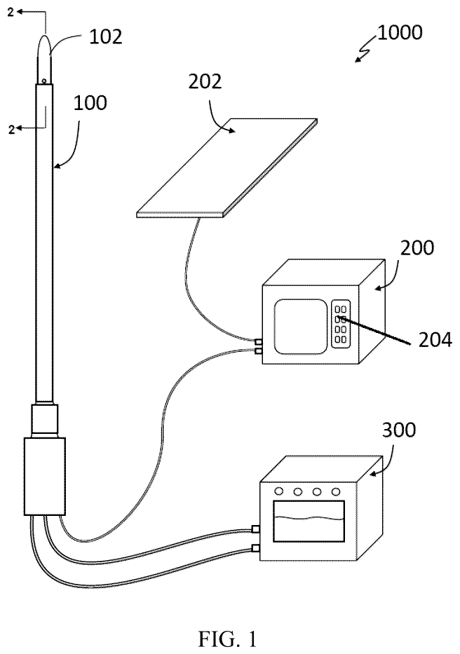

With reference to FIG. 1, an example tissue ablation system 1000 is shown. Tissue ablation system 1000 generally includes a probe 100 (also referred to as “dual mode”, “bimodal”, or “multi-mode” probe) having an active distal region 102, for delivering ablative energy to target tissue. Tissue ablation system 1000 further includes a radiofrequency (RF) generator 200 for delivering the ablative energy to the active distal region 102 of the probe 100, an external return electrode in the form of a grounding pad 202, for returning a return current from the active distal region 102 to the radiofrequency generator 200, and a fluid circulation system 300 for delivering cooling fluid to and from probe 100. (Note, while not illustrated in FIG. 1, that targeted tissue is the medium between the active distal region 102 and the grounding pad 202.) In other embodiments, tissue ablation system 1000 does not include fluid circulation system 300. In some embodiments, probe 100 does not comprise cooling features described herein.

RF generator 200 can be, for example, a generator that outputs 50 W to 300 W, that is usable with a fluid circulation system 300, and that has a control system capable of modulating the RF energy output based on a measurable parameter of the probe 100 (e.g. temperature) and/or system (e.g., impedance, current, voltage, etc.). The grounding pad 202 can be any suitable grounding pad usable with the RF generator 200. The fluid circulation system 300 can be any cooling fluid circulation system usable with the RF generator 200 and probe 100.

In some embodiments, tissue ablation system 1000 includes a multi-port hub (not shown), where probe 100 connects the hub and hub connects to generator 200.

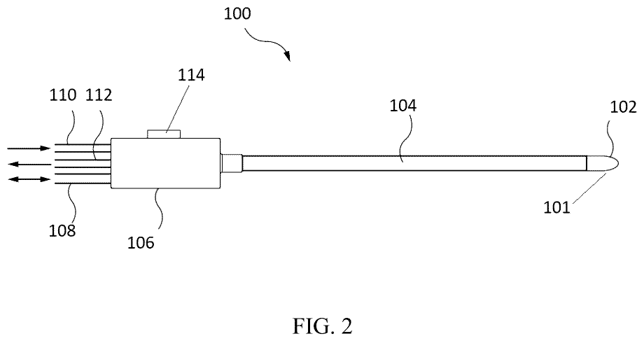

With reference to FIG. 2, in one embodiment, probe 100 has a distal end 101 and a handle 106 at its proximal end which accommodates an electrical connection to the RF generator 200. Shaft 104 proximal end is coupled to handle 106. Generator 200 is connected to the probe handle 106 by wire 108. Generator 200 supplies probe 100 with high frequency energy such as radiofrequency electrical energy which is transmitted to the active distal region 102 at the target site. The high frequency energy heats the tissue at the target site and forms a lesion by thermal ablation. Probe 100 comprises an elongate member or shaft 104. Probe 100 is insulated along its shaft 104 to prevent ablation outside of the target site. Probe 100 is configured to be inserted through the skin to the target site where soft tissue is to be lesioned.

In one embodiment, probe 100 comprises a non-conductive shaft 104 and energy is delivered via a conductive wire(s) (not shown), coupled to a plurality of active portions located on the probe 100. In such an embodiment, the conductive wire would couple the active portions (e.g., electrodes) to the RF generator 200.

In one embodiment, handle 106 provides a connection to a fluid circulation system 300 through cooling tubes 110 and 112. Shaft 104 (also referred to as “elongate member”) defines a lumen for circulating fluid, described herein. Handle 106 further comprises a switching mechanism including at least one user-selection switch that allows a user to select a mode of operation from the plurality of energy delivery modes. In one embodiment, switch 114 allows a user to enabling/disabling delivery of RF energy and/or switch between one or more modes, for example monopolar or bipolar. In other embodiments, switch 114 is not located on probe 100, for example located on generator 200. For explanatory purposes, probe 100 may be referred to as “bimodal” regardless of the number of modes available.

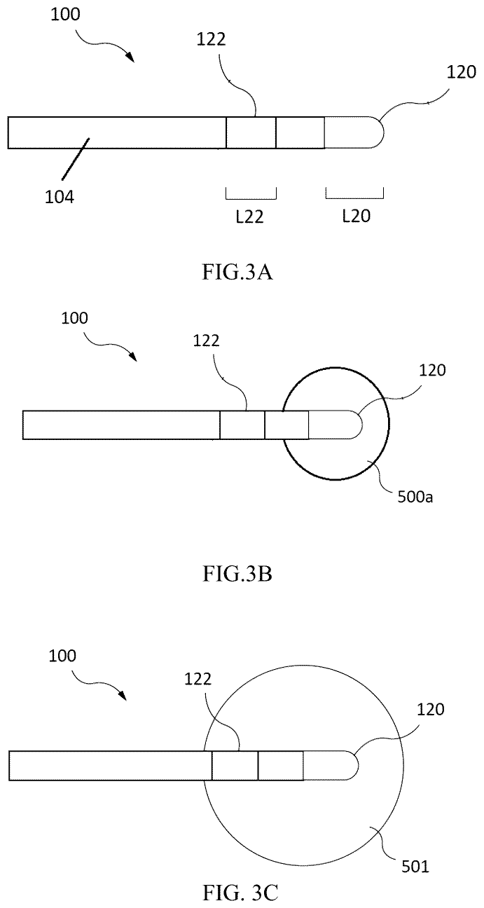

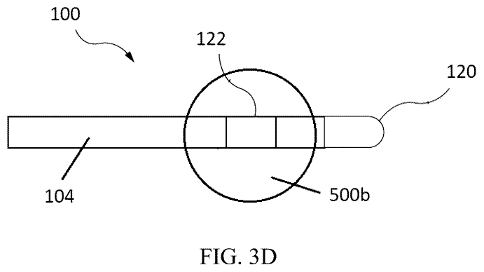

FIG. 3A shows a close-up view of active distal region 102. In one embodiment, active distal region 102 comprises a first electrode 120 and a second electrode 122. Second electrode 122 may be positioned along shaft 104 proximal to distal electrode 122 forming a coaxial, bipolar, probe.

In a monopolar mode, generator 200 delivers energy through one of the two electrodes, and the probe 100 is used with a return electrode such as a grounding pad 202. The return electrode is placed in conductive contact with skin and is connected to generator 200. The return electrode completes the electrical circuit for the high frequency energy through the patient's body.

In a bipolar mode, energy is delivered through one active electrode and the second active electrode creates a return path for the energy. An ablation zone is formed between the two electrodes.

In one embodiment, while in a bipolar mode, probe 100 can produce a larger ablation zone (by volume) than in a monopolar mode. FIG. 3B shows ablation zone 500a created using first electrode 120 in monopolar mode, and FIG. 3C shows a larger ablation zone 501 created in bipolar mode, with energy flowing from first electrode 120 to second electrode 122. In other words, volume of ablation zone 501 is greater than volume of ablation zone 500a.

Where the active monopolar electrode is the proximal of the two electrodes, this configuration ensures there is limited to no distal projection of the ablation zone beyond the tip of the electrode (which is desirable as the tip is visible under ultrasound imaging and can be used as a safety reference to demarcate the intended ablation zone). FIG. 3D shows ablation zone 500b created using electrode 122 in a monopolar mode. In some embodiments, volume of ablation zones 500a differs from volume of ablation zone 500b.

Each of monopolar mode and bipolar mode deliver RF energy has a different “energy delivery profile”. As known to those skilled in the art, “energy delivery profile” means a measure of at least one energy related parameter over a time period. The energy related parameter may be, for example, temperature, voltage, current, impedance and/or power.

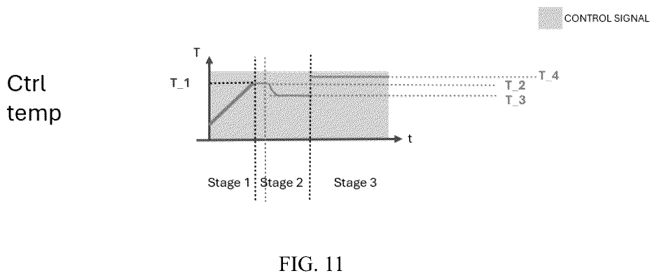

FIG. 11 shows an energy delivery profile for a radiofrequency probe during a various stages of an ablation, showing temperature over time.

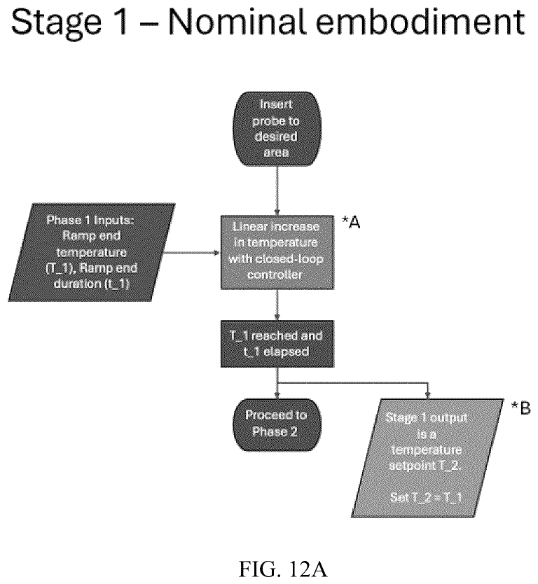

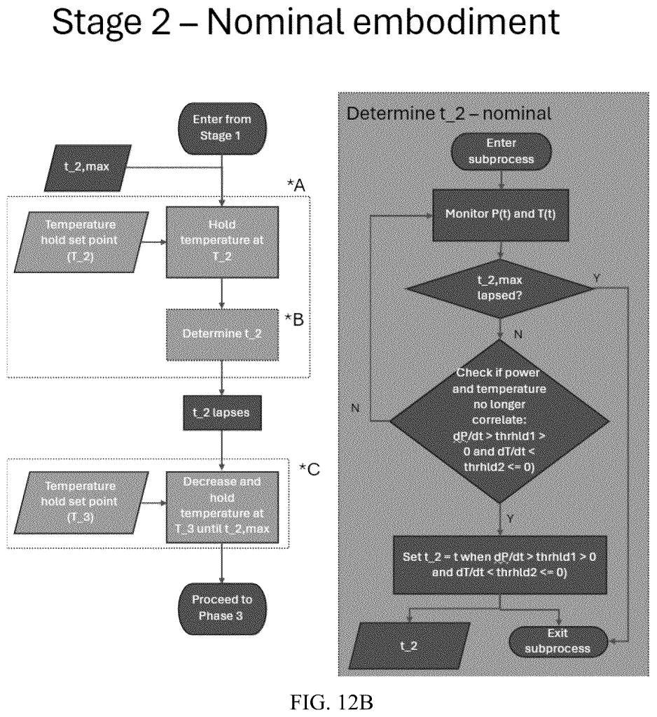

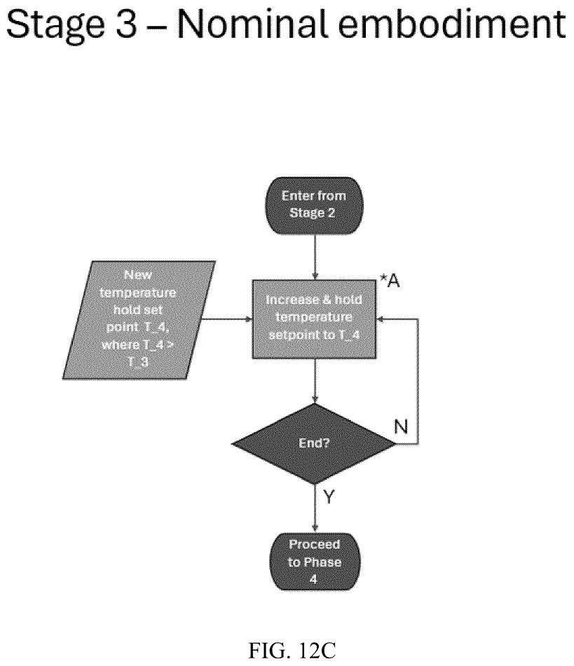

FIGS. 12A to 12C are the flow charts showing the steps of each stage of an ablation procedure.

Additionally, different energy delivery profiles may be observed within the same mode, for example, using two different electrodes having different sizes and/or different locations along the probe. In one example, a small electrode located at a distal end will produce a different energy delivery profile than a larger electrode located more proximal from the distal end, when a parameter, for example temperature, is measured by a temperature sensor located at the distal end. If the desire is to achieve a specific temperature value after a specific ramp up period, then the larger electrode will require greater energy to do so as compared to the smaller electrode. Of course, if one were to compensate for a small electrode with a high temperature, and at the same time set a low target temperature for a large electrode, the ablations size could be larger for the smaller electrode. For a given set of parameters, it is generally true that a bipolar configuration will result in larger ablations, but not always the case.

Changing the energy delivery profile from monopolar to bipolar, and vice-versa, has the effect of changing not only the size of the ablation, but also the form and location of the ablation. The term “profiles” may be used to refer to profiles that are pre-set or user-set and are used to control and regulate energy output, and also to describe energy delivery profiles which are measurands or the output of the desired pre-set or user-set profile, as would be understood by a person skilled in the art.

Switch 114 is used to switch between one or more modes of operation of probe 100. For example: at least one monopolar mode and at least one bipolar mode. In some embodiments, switch 114 is used to select which electrodes are used as the active electrodes. In such an example, probe 100 has at least two monopolar modes, and either first electrode 120 or second electrode 122 may be used as the active electrode. Switch 114 is used to select a return electrode, for example, second electrode (bipolar mode) or grounding pad 202 (monopolar mode).

In one embodiment, generator 200 comprises a switching mechanism (not shown), which operates independent of the user-selectable switch 114. In operation, switch 114 acts as a switch that notifies the generator 200 that the user intends to switch the radiofrequency output profile. The switching mechanism in the generator facilitates the switch of the RF return pathway, in other words diverts energy flow between electrodes. In one embodiment, the switch that the user actuates is the same as the switching mechanism in the generator, but in other embodiments these are two separate switches. For example, when the generator is powered on and actively delivering RF energy, the option to switch the energy delivery mechanism may be prohibited. In other words, generator 200 is in communication with probe 100, and generator 200 configures or “instructs” probe 100 to operate in a certain mode.

In one embodiment, first and second electrodes 120, 122 are substantially the same size. In another embodiment, first electrode 120 has a first size and second electrode 122 has a second size, for example, a smaller distal electrode and a larger proximal electrode. In other words, each electrode may have a different surface area, and may be defined. Such an embodiment would allow the device to produce a smaller distal ablation and a larger proximal ablation, both in the monopolar mode. Different sized electrodes give a physician the option to create a small sized distal ablation using the distal electrode in a monopolar mode, a medium sized proximal ablation using the proximal electrode in a monopolar mode, and a large size ablation using both electrodes in a bipolar mode. In some examples, electrode lengths L20 and L22 are between about 5 mm and about 7 mm. In one specific example, both the proximal and distal electrode are 7 mm, and separate by 6 mm. In another specific example, the electrodes are 5 mm and separated by 4 mm. In some examples, as L20/L22 increase, surface area increases.

In operation, in some examples, the radius of the monopolar ablation may be roughly equal to the electrode dimension. For a bipolar application, the axial dimension may be roughly 1.1× the distance from the proximal edge of the proximal electrode to the distal edge of the distal electrode; the radial dimension (radius) may be roughly 0.75× the total length of the combined electrodes.

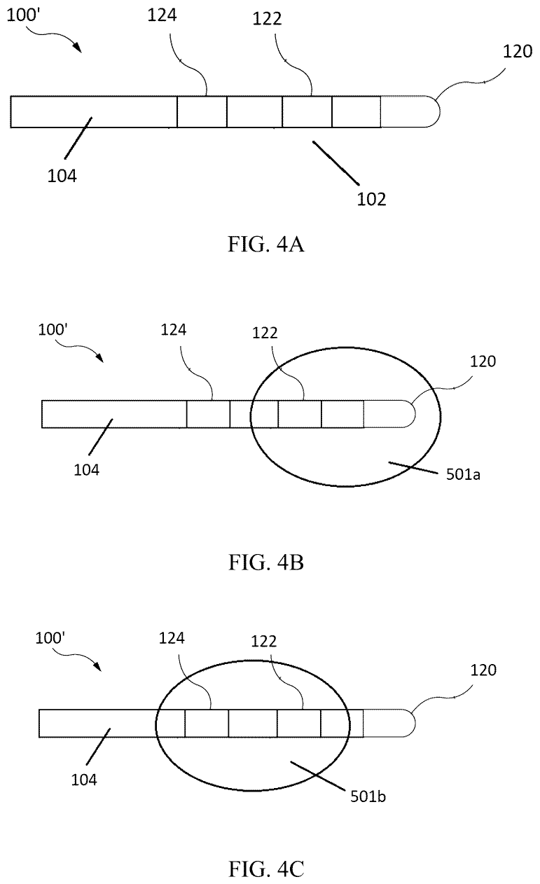

Referring to FIG. 4A, in another embodiment, probe 100′ has an active distal region 102 comprising three electrodes: a first electrode 120, a second electrode 122, and third electrode 124. These also may be referred to as distal electrode, intermediate electrode, and proximal electrode.

In some embodiments, the electrode 120, 122, 124 are substantially the same size. In another embodiment, first, second and third electrodes 120, 122, 124 are different sizes, for example a small distal electrode and a large proximal electrode, and the intermediate electrode sized between the other two. In some embodiments, the space or distance between electrodes can vary and can be more than what is depicted in the figures, in order to create oblong ablations or even two independent ablation zones. In some embodiments, the electrodes can be spaced even closer resulting in more spheroidal ablation shapes.

Switch 114 allow a user to toggle between at least two bipolar modes. In a first bipolar mode, the active electrodes are first electrode 120 and second electrode 122 (distal-intermediate). In a second bipolar mode, the active electrodes are second electrode 122 and third electrode 124 (intermediate-proximal). In other embodiments, probe 100 comprises more than three electrodes (not shown). In some embodiment, switch 114 allows a user to select which electrode acts the active electrode and a which electrode acts as the return electrode.

FIG. 4B shows an ablation zone 501a created in a first bipolar mode, and FIG. 4C shows an ablation zone 501b created while in a second bipolar mode. The ablation zone 501b in the second bipolar mode being further from the distal end 101 of probe 100′. In some embodiments, it is possible to activate any combination of electrodes in bipolar, monopolar or multipolar modes. (multipolar modes being the distal electrode and proximal electrode both connected to return intermediate electrode.) In one embodiment, switch 114 allows a user to select each individual electrode as ON(monopolar)/ON(bipolar)/OFF. In some embodiments, a user may change which electrodes are active during energy delivery, in order to create unique and asymmetric ablation volumes.

In some configurations, the change from monopolar to bipolar has the effect of the RF generator modifying the RF delivery profile, such that the rate of temperature increase, power output, maximum allowable impedance, or some other parameter is controlled or monitored in a way that is different than when the probe is in the bipolar energy delivery mode. Additionally, as the energy delivery profiles for RF ablation of thyroid nodules usually involves substantial power delivery, it is common for the electrodes to be internally cooled. As such, in some embodiments, where the probe is cooled via circulating cooling fluid, switching from monopolar mode to bipolar mode triggers a change in the internal coolant rate.

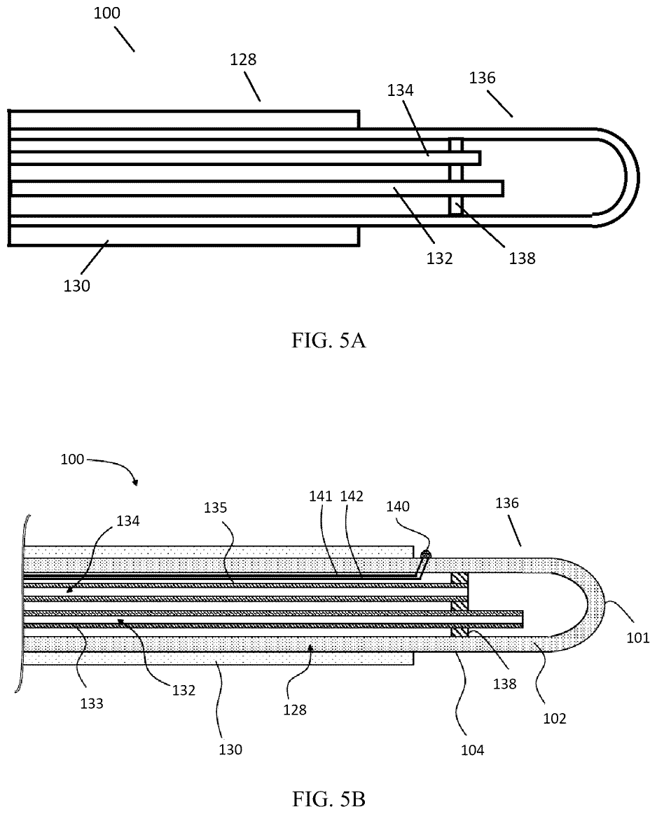

FIG. 5A is a cross section taken along line 2-2 in FIG. 1. In one embodiment, shaft 104 defines a lumen extending from the proximal end (not shown) to the distal end 101. Probe 100 includes a fluid delivery lumen 132 for delivering cooling fluid through the shaft 104 to a cooled section 136, and a fluid return lumen 134 for delivering the cooling fluid out of the cooled section 136 and proximally out of the shaft 104. In the example shown, the fluid delivery lumen 132 is provided by a first hypotube 133 and the fluid return lumen is provided by a second hypotube 135. The first and second hypotubes 133, 135 can be connected to cooling tubes 110 and 112, which in turn are connected to the fluid circulation system 300 (as shown in FIG. 1). The delivery of the cooling fluid through to the cooled section 136 cools at least a portion of the active distal region 102.

In some embodiments, the shaft 104 is configured so that on delivery of the cooling fluid (i.e. upon the exit of the cooling fluid from the fluid delivery lumen 132 and prior to the entry of the cooling fluid into the fluid return lumen 134), the cooling fluid is restricted to the cooled section 136. In some embodiments, shaft 104 includes barrier 138 to define cooled section 136. Barrier 138 restricts the cooling fluid to the cooled section 136 on delivery of the cooling fluid. In other embodiments, probe 100 does not include a barrier 138.

Probe 100 further comprises insulation 130 covering a portion of shaft 104 and defining an insulated region 128. In one embodiment, barrier 138 is positioned distally of the insulated region 128. In other embodiments, barrier 138 is located within the insulated region 128 or at the distal end of the insulated region 128.

In one embodiment, shown in FIG. 5B, probe 100 includes at least one temperature sensor 140. Temperature sensor 140 is used to measure the tissue temperature in any energy delivery mode described herein. The temperature sensor 140 can be, for example, a thermocouple junction, and the probe 100 can further include a pair of electrical conductors 141, 142 for electrically connecting the thermocouple junction to a temperature control system (which can be built into the generator 200). In alternative examples, temperature sensor 140 can be another type of temperature sensor, such as thermistor, a thermometer, or an optical fluorescence sensor. In the example shown, the electrical conductors 141, 142 extend through a lumen of the shaft 104.

Temperature sensor 140 is positioned to measure a temperature in a heat affected zone of the target tissue, and more specifically, in the thermal ablation zone. That is, the temperature sensor 140 is not internal to the shaft 104, but rather, is external to shaft 104 so that in use it is in contact with the target tissue and is sufficiently close to the active distal region 102 to measure the temperature in the thermal ablation zone.

In one embodiment, temperature sensor 140 is positioned proximally of the distal end 101 of the probe 100. Temperature sensor 140 may be spaced distal to barrier 138, in line with barrier 138 or proximal to barrier 138.

In other embodiments, the temperature sensor 140 is affixed/secured to one of the electrodes 120, 122, or 124., for example by laser welding. In some embodiments, temperature sensor 140 is located on the electrode that is configured for monopolar RF delivery, as therefore both modes have a temperature sensor associated with an energy delivery electrode.

In other embodiments, probe 100 comprises multiple temperature sensors 140, for example a first temperature sensor 140a associated with electrode 120 and a second temperature sensor 140b associated with electrode 122. In such an embodiment, either the average, maximum, or minimum of the two sensor readings can be used to control RF delivery. In other embodiments, probe 100 comprises one or more additional temperature sensors at other locations internal to the shaft 104 and/or on an external portion of the shaft 104. In some embodiments, either electrode, or both, or none can have a temperature sensor associated with it (either on the probe or a secondary sensor that is used to control, regulate, or measure the ablation progress).

In the case of “double monopolar” bimodal, where both electrodes deliver energy between the electrode and a return electrode (grounding pad), a thermocouple on each electrode can be used to independently control RF energy output per electrode.

In some embodiments, generator 200, in combination with a temperature control system (not shown), can automatically activate or deactivate the cooling, and/or adjust the cooling rate (flow rate), in response to measured temperature values in the ablation zone. For example, when certain threshold temperatures are met, (i.e., a measured temperature has increased to pre-determined temperature value) cooling may turn on or turn off, or the flow rate may be modified automatically by the generator 200. In other embodiments, cooling may be manually activated, deactivated, or flow rate may be manually modified by a user using one or more switches, described herein.

In other embodiments, for example where probe 100 does not include a temperature sensor, pulsed field ablation using high voltage, short time duration pulses are delivered. In still other embodiments, a power control algorithm is used in place of a temperature sensor feedback mechanism.

In some embodiments, generator 200 may activate or deactivate cooling, and/or modify the cooling rate in response to change in mode, for example switching from a monopolar mode to a bipolar mode. Generator 200 may also turn cooling on or off when RF energy delivery is started or stopped.

For explanatory purposes, probe 100/100′ may have at least the following modes of operation: monopolar, bipolar, or multipolar. In multipolar mode, in one example, electrodes 120 and 124 are active, and electrode 122 is the return electrode. Any mode can be used with or without cooling features. Additionally, various electrodes may be selected to use multiple monopolar or bipolar configurations, each configuration being a separate mode. E.g., first monopolar mode using active electrode 120, second monopolar mode using active electrode 122.

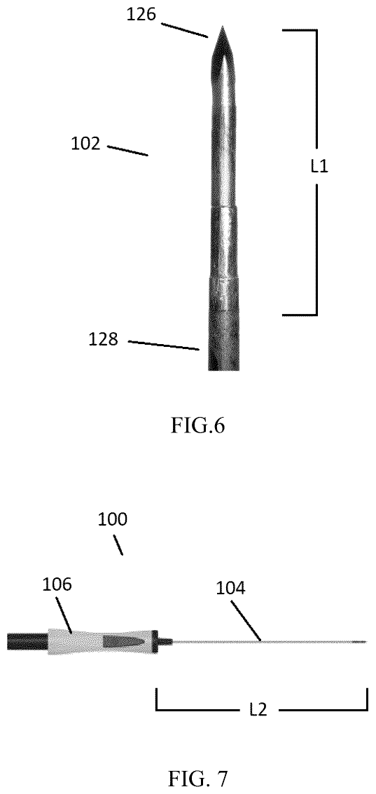

Referring to FIG. 6, in one embodiment, probe 100 comprises a sharp tip, such as a three or four-sided pyramidal shape, also referred to as a “trocar tip”, to help facilitate delivery of the probe 100, through a patient's skin, to the treatment location. In one embodiment, trocar tip 126 comprises radiopaque features so it is visible using one or more imaging modalities. In other embodiments, shaft 104 comprises radiopaque features (not shown) at other locations, for example marker bands.

Still referring to FIG. 6, active distal region 102 spans a length of L1. In some examples, L1 is between about 5 mm and about 15 mm. In some embodiments, trocar tip 126, active distal region 102, and/or other portions of the shaft 104 comprise one or more echogenic features. Echogenic features may be either passive (features on the electrode surface that reflect ultrasound energy back to the transducer), or active (an energy source is coupled to the electrode that sends acoustic energy that can be detected by the transducer of an ultrasound imaging device).

Referring to FIG. 7, in one embodiment, shaft 106 has a length L2. In some examples, L2 is between about 7 cm and about 15 cm.

Alternatively, or in addition to probe 100 having switch 114, in some embodiments, generator 200 comprises controls including one or more switches 204 (or buttons), which may be used to control one or more modes of operation of probe 100, as shown in FIG. 1. In one example, switches 204 toggle between monopolar and bipolar modes, and turns cooling on or off. In another example, a first switch 204a activates or deactivates RF energy delivery, a second switch 204b toggles the probe 100 between one more monopolar modes and one or more bipolar modes, a third switch 204c activates or deactivates a cooling mode, and a fourth switch 204d adjusts a cooling rate. Any combination of on/off/switching functions may be controlled by the handle switch 114 and/or switches 204 on the generator 200.

In some embodiments, in addition to any switches described herein, one more switches may be part of a separate device, for example a foot pedal, (not shown), connected to the generator 200 and/or probe 100.

In some embodiments, shaft 104 of probe 100 includes irrigation features where a fluid is ejected through one or more orifices (not shown) into or near the target tissue. The fluid may be the same fluid used to cool the probe.

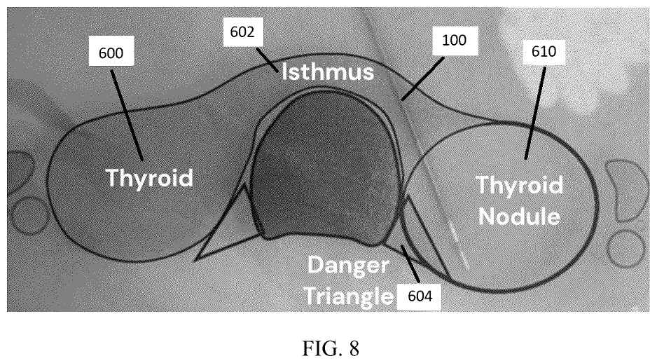

Referring to FIG. 8, probe 100 is shown in a representation of use in the human body to ablate a thyroid nodule. Thyroid 600 includes isthmus 602 and thyroid nodule 610, and a danger triangle 604, which is a region where ablation should be avoided as it contains sensitive structures such as the dorsal edge of the thyroid gland, the lateral tracheal wall, and the anterior edge of the esophageal wall, the recurrent laryngeal nerve, or the esophagus. This can sometimes be known as the “danger areas” at more caudal levels but should be understood to have similar considerations for safety. A physician typically wants to ablate tissue within thyroid nodule 610 and not any tissue within danger triangle 604. Probe 100 is shown after having been inserted, through the isthmus 602, into thyroid nodule 610.

### Method of Ablating a Nodule in a Thyroid

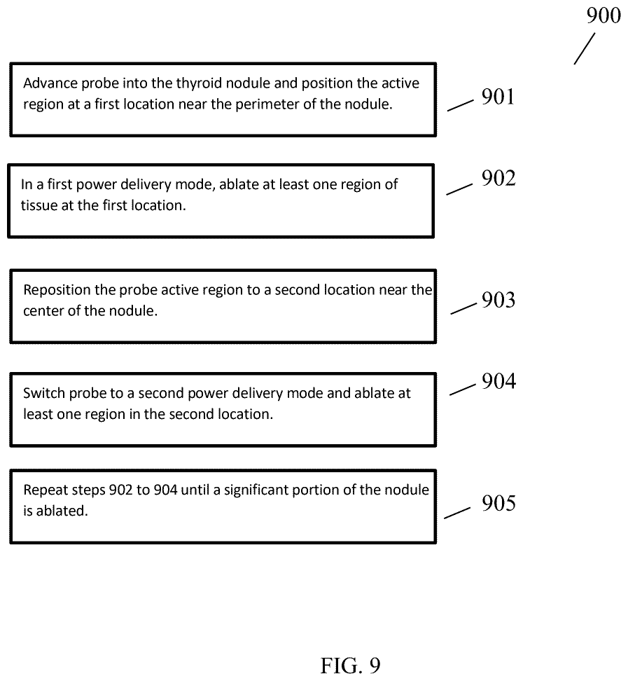

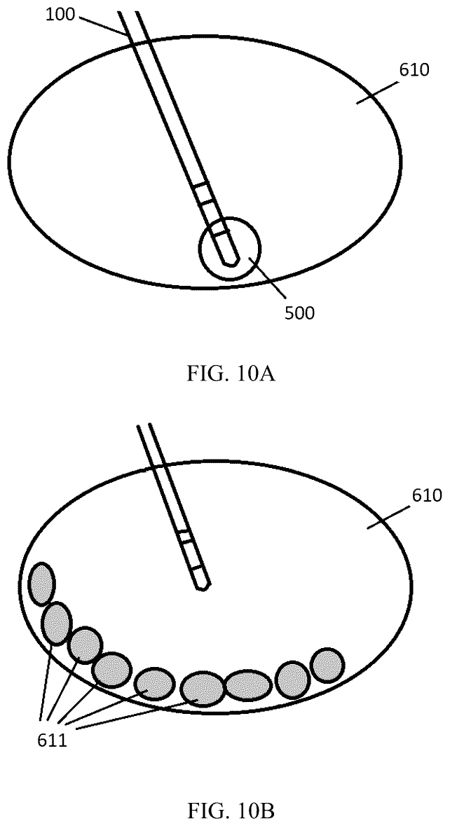

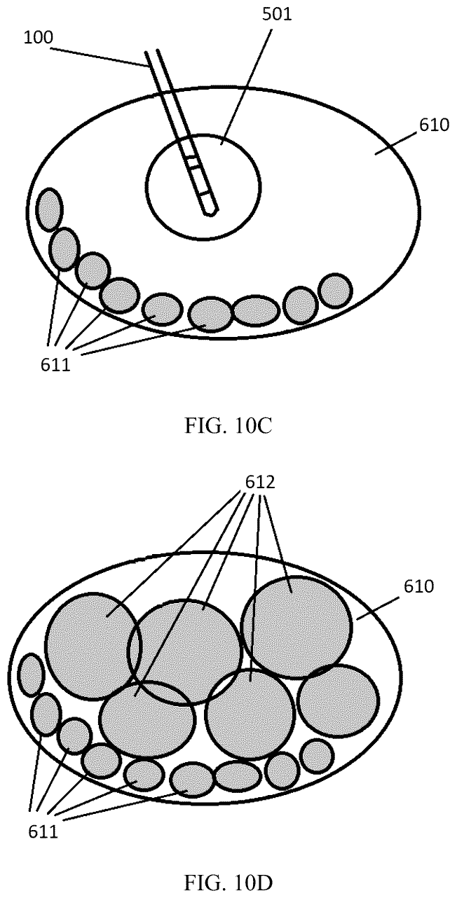

FIG. 9 illustrates a method for delivering energy to ablate a tissue using the bimodal ablation probe 100 described herein. By way of example only, the procedure described involves ablating a thyroid nodule, however the same method may be used to ablate other types of tissue, described herein. The method may be better understood with reference to FIGS. 10A to 10D.

At step 901, under a known imaging technique, the probe 100 is inserted into the thyroid 600, through the isthmus 602, and into the thyroid nodule 610.

At step 902, a physician selects a first mode, for example a monopolar mode and delivers RF energy to create lesion/ablate a region of tissue. In a monopolar mode, probe 100 creates an ablation zone 500 of a first size, as shown in FIG. 10A. In one example, the tissue being ablated is around or near the perimeter of thyroid nodule 610. In some embodiments, the physician selects active and return electrodes.

The physician may use any number of monitoring parameters to determine when this particular ablation is complete, for example when a target parameter has been reached. For example, energy could be delivered for a predetermined time, or when a measured impedance increases to a known amount, when a certain current is measured, or after a certain number of Joules have been delivered. Alternatively, or in addition to above, a physician may use any number of known imaging techniques to visualize when the ablation is complete. Acoustic properties or echogenic changes may also be measured by ultrasound.

In some embodiments, cooling features described herein are used to cool a portion of the probe 100. For example, cooling may be used during RF delivery and automatically halted when RF energy delivery ceases. Cooling may be manually activated or automatically triggered by the generator 200/fluid circulation system 300 in response to a measured temperature value as described herein.

At step 903, after one or more smaller ablated regions 611 of tissue around the perimeter have been created, the physician stops the delivery of RF energy and moves the probe 100 towards the middle of the thyroid nodule 610, a distance away from the periphery, shown in FIG. 10B.

At step 904, the physical switches to a second mode, for example a bipolar mode, and delivers RF energy to ablate a region of tissue. The ablation zone 501 of the second mode being larger (in volume) than the ablation zone 500/ablated region 611 of the first mode, as shown in FIG. 10C.

At step 905, the physician may repeat any of steps 902 to 904, in any order, as many times as necessary, alternating/toggling between modes to create various sized lesions at various locations until the thyroid nodule 610 is substantially ablated. The procedure ends when a significant portion/percentage of the thyroid nodule 610 has been ablated. Probe 100 is then removed from the tissue.

FIG. 10D shows a thyroid nodule 610 with a number of different sized ablations including smaller ablated regions 611 and larger ablated regions 612, created by delivering energy using at least two RF modes/energy delivery profiles. This includes overlapping ablation volumes.

In addition to thyroid nodule ablation, the devices and method described herein can be used in any ablation procedure where the ablation is preferred to grow along the axis of the shaft, or where multiple ablations are required. For example, liver tumor ablation or other tumor ablations where precise and well controlled ablations at the margins of the tumor are important. Applications such as Basivertebral nerve (BVN), pain management, and other applications where large ablations are required sometimes near sensitive structures would also benefit from this approach.

The embodiment(s) of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation, or identification of any reference in this application shall not be construed as an admission that such reference is available as prior art to the present invention.

1. A method of delivering energy to tissue using a radiofrequency probe, the radiofrequency probe operable between a plurality of modes, the method comprising the steps of:
delivering radiofrequency energy to create a first ablation zone within a region of tissue while the radiofrequency probe is in a first mode of the plurality of modes; and
delivering radiofrequency energy to create a second ablation zone within the region of tissue while the radiofrequency probe is in a second mode of the plurality of modes;
wherein a first ablation zone volume differs from a second ablation zone volume.

2. The method of claim 1, wherein the first mode is a monopolar mode, and the second mode is a bipolar mode.

3. The method of claim 1, wherein the method further includes a step of cooling a portion of the radiofrequency probe.

4. The method of claim 3 further comprising the steps of:
measuring a temperature value, and
automatically cooling the portion of the radiofrequency probe when the temperature value has reached a pre-determined value.

5. The method of claim 1, wherein while in the first mode, the radiofrequency energy has a first energy delivery profile and while in the second mode the radiofrequency energy has a second energy delivery profile.

6. The method of claim 5, wherein the first energy delivery profile creates an ablation zone having a first parameter, and the second energy delivery profile creates an ablation zone having a second parameter, the first parameter different from the second parameter, and wherein the first and second parameters are selected from a group consisting of size, form, and location.

7. The method of claim 1, wherein the first mode is selected from the group consisting of a first monopolar mode and a first bipolar mode, and wherein the second mode is selected from the group consisting of a second monopolar mode and a second bipolar mode.

8. The method of claim 1, further comprising a step of selecting an active electrode and selecting a return electrode.

9. The method of claim 1 wherein delivering radiofrequency energy in either mode is performed until a target parameter has been reached.

10. The method of claim 9, wherein, the target parameter is selected from a group consisting of time, impedance, current and a number of Joules delivered.

11. The method of claim 1 further comprising a step of toggling between the first and second modes at least twice.

12. The method of claim 1 wherein the region of tissue is a thyroid nodule.

13. The method of claim 12, wherein an ablation zone created in one of the first mode or the second mode is located substantially around a perimeter of the thyroid nodule, and wherein an ablation zone created in the other mode is located substantially at a distance from a periphery of the thyroid nodule.

14. A bi-modal radiofrequency ablation probe capable of operating in a plurality of energy delivery modes, the bi-modal radiofrequency ablation probe comprising:
an elongate member;
at least two electrodes associated with the elongate member; and
a switch for directing flow of radiofrequency energy through the at least two electrodes;
wherein, in use, the probe is operable to be electrically coupled to an external return electrode and the switch is operable to direct radiofrequency energy to flow in one of a plurality of energy deliver modes;
and wherein, in a first mode of the plurality of energy delivery modes, energy is directed to flow between the at least two electrodes associated with the elongate member, and, in a second mode of the plurality of energy delivery modes, energy is directed to flow between one of the at least two electrodes associated with the elongate member and the external return electrode.

15. The ablation probe of claim 14, wherein the elongate member defines a distal end and a proximal end, and a lumen defined substantially therebetween, and in use, the lumen is capable of circulating a cooling fluid through a portion of the lumen.

16. The ablation probe of claim 14, further comprising a handle, and wherein the switch is located on the handle.

17. The ablation probe of claim 14, wherein a first electrode of the at least two electrodes has a first surface area, and a second electrode of the at least two electrodes has a second surface area, and wherein the first and second surface areas differ.

18. The ablation probe of claim 14 further comprising at least one temperature sensor.

19. The ablation probe of claim 18 further comprising at least two temperature sensors.

20. The ablation probe of claim 19 wherein each of the at least two temperature sensors is associated with one electrode of the at least two electrodes.
